(19) United States
(12) Patent Application Publication
(10) Pub. No.: US 2025/0361492 A1
(43) Pub. Date: Nov. 27, 2025

(54) BACTERIOPHAGE HAVING ANTIBACTERIAL ACTIVITY AGAINST CRONOBACTER SAKAZAKII, METHOD FOR PREPARING ENCAPSULATED BACTERIOPHAGE POWDER, BACTERIOPHAGE POWDER AND COMPOSITION COMPRISING SAME

(71) Applicant: KOOKMIN UNIVERSITY INDUSTRY ACADEMY COOPERATION FOUNDATION, Seoul (KR)

(72) Inventors: Yoonjee CHANG, Seoul (KR); Seul Gi YIM, Bucheon-si Gyeonggi-do (KR)

(73) Assignee: KOOKMIN UNIVERSITY INDUSTRY ACADEMY COOPERATION FOUNDATION, Seoul (KR)

(21) Appl. No.: 19/188,364

(22) Filed: Apr. 24, 2025

Related U.S. Application Data
(63) Continuation of application No. PCT/KR2023/000304, filed on Jan. 6, 2023.

(30) Foreign Application Priority Data
Oct. 26, 2022 (KR) 10-2022-0139546
Nov. 29, 2022 (KR) 10-2022-0163013

(51) Int. Cl. C12N 7/00; A23B 2/729

(52) U.S. Cl. CPC C12N 7/00; A23B 2/7295; C12N 2795/10321; C12N 2795/10331

(57) ABSTRACT

The present disclosure relates to a novel bacteriophage having specific antibacterial activity against Cronobacter sakazakii and a method for preparing an encapsulated bacteriophage powder. The novel bacteriophage of the present disclosure exhibits high specificity for Cronobacter sakazakii, excellent lytic activity, rapid and sustained antimicrobial activity, and superior stability with respect to temperature and pH. In addition, according to the present disclosure, by encapsulating the bacteriophage using collagen peptide, the survivability of the bacteriophage can be improved. The encapsulated bacteriophage thus prepared remains stably viable even after being converted into powder form, maintains excellent antimicrobial activity, and offers enhanced convenience in distribution and use, thereby demonstrating high industrial applicability.

### CROSS-REFERENCE TO RELATED APPLICATIONS

This application is a continuation of International Application No. PCT/KR2023/000304 filed on Jan. 6, 2023, which claims priority to Korean Patent Application No. 10-2022-0139546 filed on Oct. 26, 2022 and Korean Patent Application No.10-2022-0163013 filed on Nov. 29, 2022 the entire contents of which are herein incorporated by reference.

### TECHNICAL FIELD

The present disclosure relates to a novel bacteriophage having an antibacterial activity against Cronobacter sakazakii, a method for preparing an encapsulated bacteriophage powder, a bacteriophage powder, and a composition comprising the same, and more particularly, to a novel bacteriophage having a specific antibacterial activity against Cronobacter sakazakii, a method for preparing a bacteriophage powder by encapsulating the bacteriophage with collagen peptides, a bacteriophage powder prepared thereby, and an antimicrobial composition comprising the same.

### BACKGROUND ART

Cronobacter sakazakii is a Gram-negative rod-shaped bacterium belonging to the family Enterobacteriaceae, and is a facultative anaerobe that does not form spores. It has been detected in a wide range of environments such as soil and water, as well as in various food groups including meat, vegetables, and dairy products. Cronobacter sakazakii can cause diseases in individuals of all ages, but primarily induces meningitis, enteritis, and sepsis in immunocompromised neonates or infants under six months of age, with reported fatality rates ranging from 2% to 50%. Since the occurrence of a powdered infant formula food poisoning incident in the United States in the 1950s, Cronobacter sakazakii has continued to be a global concern to this day.

A general technique for controlling pathogenic bacteria such as Cronobacter sakazakii includes a method of killing the pathogenic bacteria using antibiotics. However, in the case of antibiotics, it is difficult to achieve long-term effects due to the emergence of antibiotic-resistant strains such as “superbacteria,” and thus, there has been a need for research on antimicrobial materials capable of replacing antibiotics.

As an alternative to antibiotics, techniques utilizing natural antimicrobial materials have been developed. For example, Korean Patent Publication No. 10-2017-0115820 discloses kefir, a natural fermentation product, as a substance exhibiting antimicrobial activity against foodborne pathogens such as Cronobacter. However, the use of natural materials has limitations in that it is difficult to secure stable antimicrobial activity under various temperature and pH conditions, and it is disadvantageous in preserving the organoleptic properties of food.

Meanwhile, a technique utilizing bacteriophages has attracted attention as a material capable of replacing conventional antimicrobial substances such as the aforementioned antibiotics and natural materials. A bacteriophage is an antimicrobial substance that is a virus using bacteria as a host and induces the death of the host bacterium by binding thereto. In particular, bacteriophages have a characteristic of killing only specific categories of bacteria without affecting others. For example, Korean Patent Publication No. 10-2018-0100533 discloses a bacteriophage having the ability to specifically kill Pseudomonas aeruginosa. Due to such strain-specific properties, the use of bacteriophages has the advantage of killing only target pathogenic bacteria without killing beneficial bacteria. In addition, since 2006, bacteriophages have been recognized as a safe biological material by the U.S. Food and Drug Administration (FDA), classified as “generally recognized as safe (GRAS),” and thus may be used as an antimicrobial material applicable in various fields such as food additives.

Accordingly, the development of a bacteriophage having a specific antibacterial activity against Cronobacter sakazakii may enable its effective use in various fields requiring antimicrobial activity against Cronobacter sakazakii without concerns regarding the emergence of antibiotic-resistant strains or safety issues. In addition, since bacteriophages have a drawback of reduced stability in liquid form, if they can be stably formulated into a powder, they are expected to be more usefully applied in the food industry.

### DETAILED DESCRIPTION OF THE INVENTION

### Technical Problem

Object of the present disclosure is to provide a novel bacteriophage having a specific antibacterial activity against Cronobacter sakazakii.

Another object of the present disclosure is to provide a method for preparing a bacteriophage powder exhibiting excellent survival rate of the bacteriophage.

Still another object of the present disclosure is to provide a bacteriophage powder having improved stability, prepared by the above method.

Still another object of the present disclosure is to provide an antimicrobial composition comprising the bacteriophage or the bacteriophage powder.

### Means for Solving the Problem

In order to achieve the above object, the present disclosure provides a bacteriophage having an antibacterial activity against Cronobacter sakazakii, deposited under accession number KCTC15074BP.

In the present disclosure, the bacteriophage belongs to the family Siphoviridae.

In the present disclosure, it is preferable that the bacteriophage is used at a multiplicity of infection (MOI) of 0.1 or less.

The present disclosure also provides a method for preparing a bacteriophage powder, the method comprising: (i) mixing an encapsulating solution comprising a collagen peptide and a bacteriophage to prepare a mixed solution; and (ii) drying the mixed solution to obtain an encapsulated bacteriophage powder.

In the present disclosure, the bacteriophage may be a bacteriophage having an antibacterial activity against Cronobacter sakazakii.

In the present disclosure, the bacteriophage may be a bacteriophage with accession number KCTC 15074BP.

In the present disclosure, the concentration of the bacteriophage in the mixed solution may be from 10⁵ to 10¹⁵ PFU/mL.

In the present disclosure, the concentration of the collagen peptide in the encapsulating solution may be from 0.01 to 0.2 g/mL.

In the present disclosure, the encapsulating solution may further comprise trehalose.

The concentration of trehalose in the encapsulating solution may be from 0.001 to 0.1 g/mL.

In the present disclosure, the mixed solution may comprise one or more solvents selected from the group consisting of water, methanol, ethanol, propanol, 1,3-propanediol, butanol, pentanol, hexanol, propylene glycol, dipropylene glycol, butylene glycol, glycerin, acetone, ethyl acetate, butyl acetate, chloroform, diethyl ether, dichloromethane and hexane.

In the present disclosure, step (ii) may be performed by freezing the mixed solution at a temperature below 0° C. for 3 hours or more, followed by drying for 18 hours or more.

The present disclosure also provides a bacteriophage powder prepared by the method for preparing the bacteriophage powder.

In the present disclosure, the bacteriophage powder may have a structure comprising a bacteriophage and a capsule wall, wherein the capsule surrounds the bacteriophage and comprises a collagen peptide.

The present disclosure also provides an antimicrobial composition comprising the bacteriophage or the bacteriophage powder.

### Effects of the Invention

The novel bacteriophage of the present disclosure exhibits very high specificity against Cronobacter sakazakii, excellent lytic activity, rapid and sustained antimicrobial activity, and outstanding stability with respect to temperature and pH. Furthermore, when the bacteriophage is encapsulated using a collagen peptide according to the present disclosure, the bacteriophage remains stable and viable even after being converted into powder form, exhibiting excellent antimicrobial activity, and offering high convenience for distribution and use, making it highly suitable for industrial applications.

Therefore, the present disclosure can be effectively applied to antimicrobial compositions using bacteriophages, and particularly, it can be usefully employed as an additive to prevent the growth and reproduction of Cronobacter sakazakii in foods such as powdered infant formula.

### SIMPLE DESCRIPTION OF DRAWINGS

FIG. 1 shows a transmission electron microscope (TEM) image of bacteriophage SG01isolated according to one embodiment of the present disclosure.

FIG. 2 shows the genome map of bacteriophage SG01 identified in one embodiment of the present disclosure.

FIG. 3 shows the phylogenetic tree of bacteriophage SG01 identified in one embodiment of the present disclosure.

FIG. 4 is a graph showing the results of measuring the Cronobacter sakazakii growth inhibitory activity of bacteriophage SG01 according to one embodiment of the present disclosure.

FIG. 5 is a graph showing the results of measuring the survival rate of bacteriophage SG01 in the temperature range of −18° C. to 80° C. according to one embodiment of the present disclosure.

FIG. 6 is a graph showing the results of measuring the survival rate of bacteriophage SG01 in the pH range of 3 to 12 according to one embodiment of the present disclosure.

FIG. 7a is a graph showing the results of measuring the biofilm reduction rate of bacteriophage SG01 according to one embodiment of the present disclosure.

FIG. 7b is a graph showing the results of measuring the biofilm inhibition rate of bacteriophage SG01 according to one embodiment of the present disclosure.

FIG. 8 is a graph showing the results of measuring the survival rate according to the concentration of collagen peptide of the bacteriophage powder used in one embodiment of the present disclosure.

FIG. 9 is a graph showing the results of measuring the survival rate according to the concentration of trehalose of the bacteriophage powder used in one embodiment of the present disclosure.

FIG. 10 is a graph showing the results of measuring the Cronobacter sakazakii growth inhibitory activity in powdered infant formula using the bacteriophage powder according to one embodiment of the present disclosure.

### MODES FOR CARRYING OUT THE INVENTION

Hereinafter, the specific embodiments of the present disclosure will be described in more detail. Unless otherwise defined, all technical and scientific terms used herein have the same meanings as commonly understood by those skilled in the art to which the present disclosure pertains. In general, the nomenclature used herein is well-known and commonly used in the art.

The present disclosure relates to a bacteriophage having a specific antibacterial activity against Cronobacter sakazakii.

A bacteriophage is a virus that uses bacteria as its host and can be abbreviated as “phage.” A bacteriophage kills the host bacterium through a lytic cycle and/or a lysogenic cycle. For example, according to the lytic cycle, after infecting the bacterium, the bacteriophage replicates inside the bacterial cell and, upon replication, is released by destroying the bacterial cell wall, thereby killing the bacterium. A single type of bacteriophage has antibacterial activity against only specific categories of host bacteria, so depending on the type of bacterium to be killed, either a specific bacteriophage may be selected, or a novel bacteriophage may be discovered and used.

Meanwhile, Cronobacter sakazakii is a foodborne pathogen that exists in infant formula and cereal-based prepared foods, and when contaminated in infant foods, especially in infant formula, it can cause serious diseases such as meningitis in infants, potentially leading to death in severe cases.

The present disclosure provides a novel bacteriophage having an antibacterial activity against Cronobacter sakazakii, namely bacteriophage SG01 (hereinafter referred to as “phage SG01”).

The phage SG01 is deposited at the Korean Collection for Type Culture (KCTC) with accession number KCTC15074BP (deposit date: Sep. 2, 2022).

The bacteriophage used in the present disclosure belongs to the family Siphoviridae and may exhibit specific antibacterial activity against Cronobacter sakazakii. Specifically, the bacteriophage of the present disclosure may demonstrate antibacterial activity against one or more strains selected from the group consisting of Cronobacter sakazakii ATCC 29544, Cronobacter sakazakii NCTC 2949, Cronobacter sakazakii ATCC 29004, Cronobacter sakazakii ATCC BAA-894, Cronobacter sakazakii ES15, Cronobacter sakazakii ATCC 51329, Cronobacter sakazakii isolates 1, Cronobacter sakazakii isolates 2, Cronobacter sakazakii isolates 5 and Cronobacter sakazakii isolates 6. In an embodiment of the present disclosure, when phage SG01 was infected with 36 different strains, it was confirmed that it specifically infected Cronobacter sakazakii while not causing infection in other strains.

The bacteriophage used in the present disclosure may exhibit rapid and long-lasting antimicrobial activity against Cronobacter sakazakii. In an embodiment of the present disclosure, it was confirmed that treatment with phage SG01 rapidly inhibited the growth of Cronobacter sakazakii within 1 hour, and particularly, when the phage count was low under an MOI of 0.01, the antimicrobial effect was sustained for up to 20 hours.

The bacteriophage of the present disclosure exhibits excellent thermal stability and pH stability, making it applicable under various temperature and pH conditions. Specifically, phage SG01 can survive in a temperature range of −18 to 70° C. and a pH range of 4 to 12. Notably, in the temperature range of −18 to 50° C. and the pH range of 6 to 11, the survival rate of the phage is scarcely affected by temperature and pH. As such, the bacteriophage of the present disclosure, with its high stability to temperature and pH, can be used without limitation in applications that require post-treatment and processing.

The bacteriophage of the present disclosure can effectively control the biofilm induced by Cronobacter sakazakii. Specifically, when the bacteriophage of the present disclosure is applied to the biofilm formed by Cronobacter sakazakii, it not only shows an effect of removing the biofilm, but also demonstrates an inhibitory effect in preventing the formation of biofilm by Cronobacter sakazakii.

When the bacteriophage of the present disclosure is used for bacterial killing, the antimicrobial activity of the phage may be regulated depending on the multiplicity of infection (MOI) conditions.

In one embodiment of the present disclosure, the MOI of phage SG01 may be 0.1 or less, preferably between 0.001 and 0.05. In this case, it may exert long-lasting antimicrobial effects against Cronobacter sakazakii with a small amount.

In another embodiment of the present disclosure, the MOI of phage SG01 may be 50 or more, preferably between 80 and 500. In this case, a very rapid antimicrobial effect against Cronobacter sakazakii may be observed.

As such, the bacteriophage of the present disclosure can specifically kill Cronobacter sakazakii, exhibit rapid and sustained antimicrobial activity, demonstrate excellent stability with respect to temperature and pH, and show both removal and inhibition effects on biofilm. Therefore, the bacteriophage of the present disclosure can be effectively used in various applications to induce the death of Cronobacter sakazakii or prevent infections caused by it.

The present disclosure provides a method for preparing an encapsulated bacteriophage powder and the bacteriophage powder prepared using the method.

Bacteriophages have primarily been used in solution form, however, bacteriophages in solution are prone to reduced stability during distribution, offer limited convenience for commercial use, and are difficult to apply to dry foods such as powdered infant formula. Additionally, when bacteriophages are encapsulated to enhance their stability and converted into powder, the survival rate tends to be low, and as a result, the stability improvement effect is not significant.

In the present disclosure, by encapsulating the bacteriophage with collagen peptide, the survival rate of the bacteriophage can be enhanced, and the bacteriophage can maintain excellent antimicrobial activity even after being processed into powder.

According to the present disclosure, the encapsulated bacteriophage powder may have a structure comprising a bacteriophage and a capsule wall surrounding the bacteriophage and containing collagen peptide.

In the present disclosure, a bacteriophage having an antibacterial activity against Cronobacter species (Cronobacter sp.) may be used, and preferably, a bacteriophage having an antibacterial activity against Cronobacter sakazakii may be used. Specifically, the bacteriophage SG01 may be used as a bacteriophage having an antibacterial activity against Cronobacter sakazakii. This allows for the stable application of the bacteriophage to infant foods such as powdered infant formula, thereby inhibiting the growth and reproduction of Cronobacter sakazakii and preventing infections caused by it.

Bacteriophages generally have low encapsulation efficiency, and even when encapsulated, the improvement in stability is not significant. The present disclosure overcomes this limitation by using collagen peptides as the encapsulation material for the bacteriophage.

The collagen peptide has excellent moisture retention ability, which enables the bacteriophage to exhibit a high survival rate during encapsulation. It also has excellent biocompatibility and bioaffinity, making it applicable in the food and cosmetics industries. Specifically, when low molecular weight hydrolyzed collagen peptides are applied to powdered infant formula or baby food, they enhance the digestibility and absorption rate in the body, which has the advantage of not affecting the health of neonates and infants when consumed.

In the present disclosure, the bacteriophage powder may be prepared by a method comprising: (i) mixing an encapsulation solution comprising a collagen peptide and a bacteriophage to prepare a mixed solution; and (ii) drying the mixed solution.

In the mixed solution, the concentration of the bacteriophage may be from 10⁵ to 10¹⁵ PFU/mL, preferably from 10⁶ to 10¹² PFU/mL, and more preferably from 10⁷ to 10¹⁰ PFU/mL.

In the encapsulating solution, the concentration of collagen peptide may be from 0.01 to 0.2 g/mL, preferably from 0.02 to 0.1 g/mL, more preferably from 0.03 to 0.07 g/mL, and most preferably from 0.04 to 0.06 g/mL. If the concentration of collagen peptide is lower than the above range, the yield and the weight relative to the volume of the encapsulated bacteriophage powder may significantly decrease, leading to issues such as powder scattering when applied in the food industry. Additionally, it may result in a lack of crystallinity, giving the powder a light, cotton-like appearance rather than a solid, spherical form, which is undesirable. Furthermore, if the concentration of collagen peptide is too high, it may be undesirable from the perspective of efficiency and cost-effectiveness during production.

In one embodiment of the present disclosure, it was confirmed that the bacteriophage encapsulated with collagen peptide in the concentration range exhibited superior survival rate compared to the non-encapsulated bacteriophage. Specifically, when the collagen peptide concentration was 0.05 g/mL, the bacteriophage survival rate was found to be the highest.

In a preferred embodiment of the present disclosure, the encapsulating solution may further comprise trehalose. Trehalose, a disaccharide composed of two glucose molecules, may be mixed with collagen peptide as an encapsulation material in the present disclosure, thereby further enhancing the survival rate of the bacteriophage.

In the encapsulating solution, the concentration of trehalose may be from 0.001 to 0.1 g/mL, and preferably from 0.005 to 0.05 g/mL. If the concentration of trehalose is lower than the above range, it is undesirable in terms of the efficiency of increasing the survival rate of the bacteriophage. Conversely, if the concentration of trehalose is too high, it is undesirable in terms of efficiency and cost-effectiveness during production. In this regard, even when trehalose is used in small amounts, ranging from 0.006 to 0.03 g/mL in the encapsulating solution, a very excellent survival rate enhancement effect can be achieved, making it preferable. More preferably, the same effect can be achieved with trehalose concentrations between 0.008 and 0.02 g/mL.

In the mixed solution, the amount of bacteriophage per 1 g of collagen peptide may be from 10⁵ to 10¹² PFU, and preferably from 10⁸ to 10¹⁰ PFU. The concentration of collagen peptide in the mixed solution, based on the total volume of the solution, may be from 0.01 to 0.2 g/mL, and preferably from 0.02 to 0.1 g/mL, more preferably from 0.03 to 0.7 g/mL, and most preferably from 0.04 to 0.06 g/mL.

Preferably, the mixed solution may be prepared by mixing the bacteriophage solution and the encapsulating solution. In the present disclosure, water or an organic solvent may be used as the solvent for each solution. Specifically, one or more solvents selected from the group consisting of water, methanol, ethanol, propanol, 1,3-propanediol, butanol, pentanol, hexanol, propylene glycol, dipropylene glycol, butylene glycol, glycerin, acetone, ethyl acetate, butyl acetate, chloroform, diethyl ether, dichloromethane and hexane may be used.

The concentration of the bacteriophage solution may be from 10⁵ to 10¹⁵ PFU/mL, and preferably from 10⁶ to 10¹² PFU/mL, more preferably from 10⁷ to 10¹⁰ PFU/mL.

The mixing volume ratio of the bacteriophage solution and the encapsulating solution may be from 1:10 to 1:1,000, and preferably from 1:20 to 1:200 .

The drying of the mixed solution may be performed by freeze-drying, specifically by freezing the mixed solution at a temperature below 0° C. for 3 hours or more, preferably for 12 to 72 hours, followed by drying for 18 hours or more, preferably for 24 to 96 hours. This process results in the production of encapsulated bacteriophage powder in powder form.

According to the present disclosure, the bacteriophage powder encapsulated with collagen peptide exhibits excellent stability, and especially, the bacteriophage remains stable and viable in powder form, demonstrating excellent antimicrobial activity. Furthermore, due to the biocompatibility and stability of the material itself, it can be effectively used not only in general antimicrobial agents and preservatives but also in fields requiring high safety, such as food and cosmetics.

Accordingly, the present disclosure also provides an antimicrobial composition comprising the bacteriophage or bacteriophage powder.

The term “antimicrobial composition” as used herein refers to a formulation capable of killing bacteria or inhibiting the growth of bacteria, and may collectively refer to antimicrobial agents such as preservatives, disinfectants, sterilizers, detergents, antibiotics and preservatives. Additionally, the antimicrobial composition is interpreted as including a formulation that reduces or prevents not only the bacterium itself but also the formation of biofilms formed by the bacterium, that is, an anti-biofilm agent. The formulation of the antimicrobial composition comprising the bacteriophage or its powder may be in liquid, semi-solid, or powder form.

The antimicrobial composition comprising the bacteriophage or bacteriophage powder as an active ingredient exhibits very high specificity against Cronobacter sakazakii compared to conventional antimicrobial compositions, allowing it to kill only specific pathogens without killing beneficial bacteria and without inducing drug resistance, thus showing sustained effects. Additionally, due to its excellent thermal and pH stability, it can be applied under various temperature and pH conditions.

The antimicrobial composition may comprise an adequate amount of additives sufficient to reduce the deterioration of the quality of the antimicrobial composition.

The additives may be preservatives, stabilizers, excipients, or cryoprotectants, and any substances conventionally used in the industry that may reduce the quality deterioration of the antimicrobial composition may be used without limitation.

The antimicrobial composition may be used in various applications where antimicrobial activity against Cronobacter sakazakii is required. Particularly, since Cronobacter sakazakii may have severe impacts on children under the age of six, i.e., infants, the present disclosure can provide significant antimicrobial effects in infant foods such as powdered infant formula, baby food, as well as infant products such as baby bottles, formula spoons, and bottle cleaning brushes.

Accordingly, the present disclosure also provides a food preservation additive comprising the bacteriophage or bacteriophage powder, and a food comprising the same.

By applying the food preservation additive comprising the bacteriophage or bacteriophage powder to food, the growth and reproduction of Cronobacter sakazakii in the food can be prevented, thereby improving the safety and shelf life of the food and preventing infectious diseases caused by Cronobacter sakazakii in the food.

The food preservation additive of the present disclosure may further comprise additives usable in food, such as pH regulators, stabilizers, alginate, vitamins, flavorings, colorants, etc., in addition to the bacteriophage or bacteriophage powder.

The food to which the food preservation additive of the present disclosure may be applied includes various food products, such as dairy products, processed meat products, seafood products, tofu, jellies, noodles, health supplements, seasoned foods, sauces, snacks, dairy products, pickled foods, beverages, natural seasonings, vitamin complexes, alcoholic beverages and other processed foods. The formulation of the food preservation additive comprising the bacteriophage or the food comprising the same of the present disclosure may be in liquid, semi-solid or powder form.

In particular, considering that Cronobacter sakazakii has a significant impact on infants, the use of the bacteriophage or bacteriophage powder as a food preservation additive in infant foods such as powdered infant formula and baby food can inhibit the growth and reproduction of Cronobacter sakazakii, thereby preventing the occurrence of fatal Cronobacter sakazakii infections in infants.

### EXAMPLES

Hereinafter the present disclosure will be described in more detail through Examples. However, these Examples show some experimental methods and compositions to illustratively illustrate the present disclosure, and the scope of the present disclosure is not limited to these Examples.

### Experimental Methods

In the experiment, Cronobacter sakazakii ATCC 29544 was used as the host bacterium, and TSB broth (MB-T1053;MB cell, Seoul, Korea), 0.4% (w/v) molten TSA, and 1.5% (w/v) TSA agar (MB-T1052, MB cell) were used as the culture media.

The phage titer was measured using a double-layer agar plate, with 0.5% (w/v) molten TSA and 1.5% (w/v) TSA agar as the upper and lower layers, respectively.

### Preparation Example 1: Purification, Amplification and Stock Preparation of Phage SG01

A bacteriophage (hereinafter referred to as “phage”) was obtained from an environmental sample (sewage) and purified using a double-layer agar assay and a plaque assay. A single plaque was resuspended in phosphate-buffered saline (PBS), centrifuged at 15,000×g for 1 minute at 4° C., and filtered through a sterile Whatman™ PVDF membrane filter with a pore size of 0.22 μm. This filtration process was repeated five times.

To amplify the isolated phage, the phage was cultured in LB broth using Cronobacter sakazakii ATCC 29544 as a host. Specifically, Cronobacter sakazakii ATCC 29544 was subinoculated at 1% and cultured at 37° C. for 1.5 hours. Thereafter, the phage was cultured under aerobic conditions at 37° C. for 4 hours. The sample was then centrifuged at 15,000×g for 10 minutes at 4° C., and the supernatant was filtered through a sterile Whatman™ PVDF membrane filter with a pore size of 0.45 μm. The above steps were sequentially performed under three different volume conditions (3, 50, and 300 mL of host culture) to obtain a sufficient amount of phage lysate.

To obtain a phage stock with a higher titer, the purified phage lysate was centrifuged at 30,000×g for 30 minutes at 4° C. to obtain a pellet. The phage concentration (PFU/mL) of the resulting pellet was measured using the double-layer agar assay. The purified phage was amplified to yield a lysate with a titer of 10¹⁰ PFU/mL or higher, which was stored at 4° C. until use. For long-term storage, it was stored at −80° C. in 35% glycerol.

The phage isolated and purified by the above method was designated as “phage SG01” and was deposited with the Korean Collection for Type Culture (KCTC) under accession number KCTC15074BP on Sep. 2, 2022.

### Experimental Example 1: Transmission Electron Microscopy (TEM) Analysis of Phage SG01

The morphological characteristics of phage SG01 were analyzed using transmission electron microscopy (TEM).

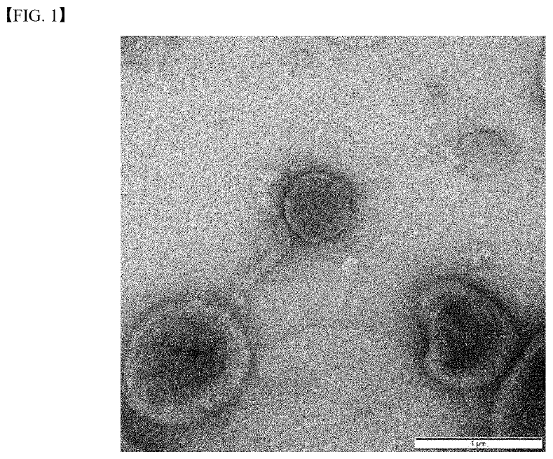

A 200-mesh copper grid coated with formvar/carbon was pretreated using an electrical discharge machine (US/91000, USA). After loading the phage onto the copper grid, negative staining was performed using 2% (v/v) uranyl acetate (pH 4.5). The sample was then analyzed using an energy-filtering Libra 120 transmission electron microscope (Carl Zeiss, Germany), and the results are shown in FIG. 1.

Referring to the TEM image of FIG. 1, it was confirmed that phage SG01 has an icosahedral head and a flexible, non-contractile tail. Specifically, the head of the phage has an icosahedral shape with a diameter of 0.66±0.05 μm, the tail length is 1.63±0.07 μm, and the tail width is 0.12±0.01 μm. From these results, it was determined that phage SG01 is classified into the family Siphoviridae.

Phage SG01 is similar to other Cronobacter sakazakii phages (e.g., phage CS01) in that it exhibits antimicrobial activity against Cronobacter sakazakii. However, based on morphological analysis, unlike CS01 phage, which has a non-flexible, contractile tail and is classified into the family Myoviridae, SG01 phage has a non-contractile, flexible tail and is classified into the family Siphoviridae.

### Experimental Example 2: DNA Analysis of Phage SG01

### Genetic Sequence Analysis and Bioinformatics Analysis

The DNA of phage SG01 was extracted, and open reading frames (ORFs) of the phage genome were identified using RAST (https://rast.nmpdr.org/), GeneMarkS (http://exon.gatech.edu/GeneMark/genemarks.cgi) and FgenesV (trained Pattern Markov chain-based viral gene prediction software). For unidentified ORFs, the ORF prediction was supplemented by referencing the non-overlapping protein NCBI database (http://blast.ncbi.nlm.nih.gov/) using BLASTP, as well as homologous ORFs from other known bacteriophages.

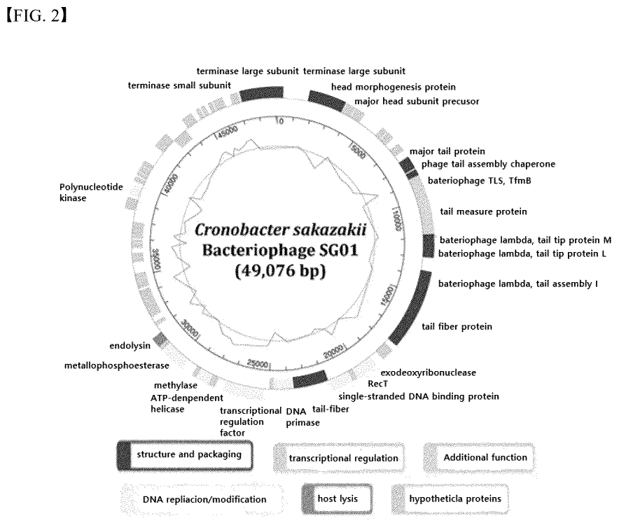

Based on the analysis results, a genome map was generated using Genescene software (DNASTAR, Madison, WI), and is shown in FIG. 2. The genome sequence of the phage was deposited in GenBank under accession number OP120783.

As a result of the analysis, the genome of phage SG01 was inferred to be composed of 49,076 bp. Among them, the GC content was found to be 50.01%, and the number of coding OR Fs was identified to be 60.

The ORF analysis revealed that the phage does not contain lysogeny module genes or toxic genes, thereby confirming the safety of the phage.

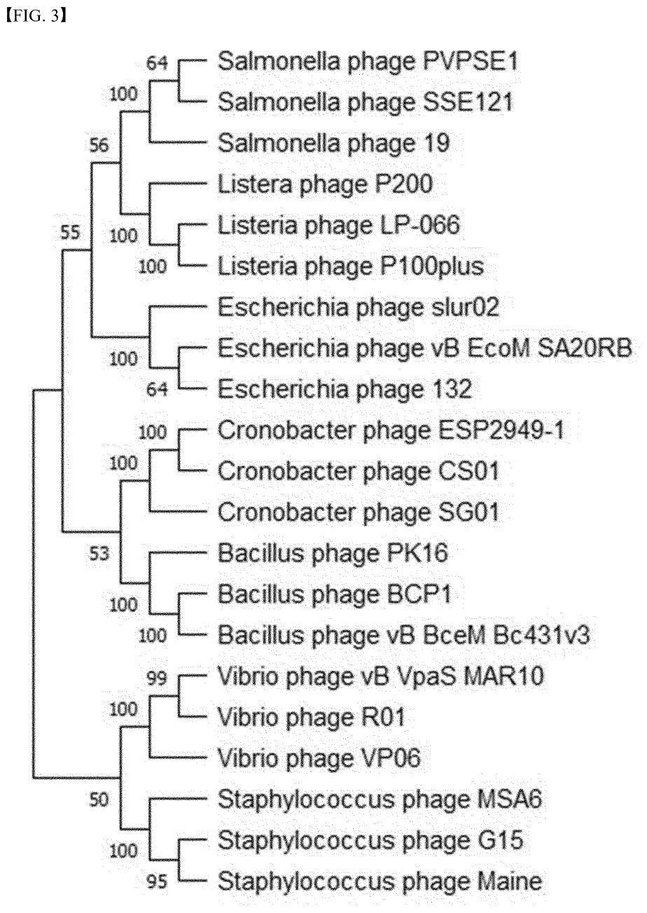

For phylogenetic identification of the phage, phylogenetic analysis based on the terminase large subunit was performed using the Molecular Evolutionary Genetics Analysis 11 (MEGA 11) software by the neighbor-joining method with 2,000 bootstrap replications. The resulting phylogenetic tree is shown in FIG. 3.

As a result of the phylogenetic analysis, the terminase large subunit of phage SG01 was found to be similar to that of other Cronobacter sakazakii phages, namely phages ESP2949-1 and CS01, thereby confirming that phage SG01 belongs to the Cronobacter sakazakii phage group.

### Experimental Example 3: Measurement of Antimicrobial Activity of Phage SG01

The antimicrobial activity of phage SG01 was evaluated by measuring the optical density after adding phage SG01 to Cronobacter sakazakii.

Cronobacter sakazakii ATCC 29544 was cultured in TSB medium for 1.5 hours. Thereafter, phage SG01 was infected into the culture at multiplicities of infection (MOIs) of 0.01, 0.1, and 1, respectively. While allowing the host to grow for 24 hours, the optical density at 600 nm was measured at 1-hour intervals using a spectrophotometer to confirm the growth inhibitory activity against Cronobacter sakazakii.

FIG. 4 shows the results of measuring the growth inhibitory activity of Cronobacter sakazakii in the presence of phage SG01. Referring to FIG. 4, in the negative control group without phage treatment, the bacteria rapidly proliferated, whereas in the groups treated with the phage, the growth of the host bacterial cells was rapidly inhibited within 1 hour under MOI conditions of 1, 0.1 and 0.01. In addition, the growth inhibition persisted for 6 hours at an MOI of 1, for 10 hours at an MOI of 0.1, and for 20 hours at an MOI of 0.01.

From these results, it was confirmed that phage SG01 exhibits rapid and long-lasting antimicrobial activity against Cronobacter sakazakii.

### Experimental Example 4: Host Range Determination of Phage SG01

A spot test was performed on the bacterial strains listed in Table 1 to determine the host range of phage SG01.

Ten microliters(10 μL) of decimal-diluted phage lysate was spotted onto the lawns of a total of 36 strains and incubated at 37° C. for 24 hours. The lytic plaque-forming efficiency of the phage was measured against Cronobacter sakazakii strains as well as several Gram-positive and Gram-negative strains, and the results are shown in Table 1 below. The efficiency of plating (EOP) was calculated according to the formula below, where “++” indicates an EOP of 0.1 to 1, “+” indicates an EOP of less than 0.1, and “−” indicates no susceptibility to the phage.

EOP=(Number of final lytic plaques on the target strain (PFU/mL)/Number of final lytic plaques on the reference strain (PFU/mL))

TABLE 1

| | Plaque |
|---|---|
| Bacterial isolate | formation |
| Salmonella enterica Enteritidis ATCC 1306 | − |
| Salmonella enterica UK1 | − |
| Escherichia coli O157 H7 ATCC 43890 | − |
| Escherichia coli O157 H7 ATCC 35150 | − |
| Kiebsiella oxytoca KCTC 1686 | − |
| kiebsiella pneumoniae KCTC 2242 | − |
| Enterococcus faecalis ATCC 19433 | − |
| Vibrio parahaemolyticus KCTC 2471 | − |
| Vibrio cholerae NCCP 13589 | − |
| Shigella sonnet KCTC 22530 | − |
| Shigella flexneri KCTC 2517 | − |
| Pseudomonas aeruginosa ATCC 27853 | − |
| Yersinia enterocolitica ATCC 55075 | − |
| Pectobacterium carotovorum KACC 21701 | − |
| Staphylococcus aureus ATCC 29213 | − |
| Staphylococcus aureus CCARM 3090 | − |
| Listeria monocytogenes ATCC 15313 | − |
| Listeria innocua KCTC 3586 | − |
| Bacillus cereus ATCC 27348 | − |
| Bacillus cereus ATCC 14579 | − |
| Cronobacter sakazakii ATCC 29544 (Host) | ++ |
| Cronobacter sakazakii NTCT2949 | ++ |
| Cronobacter sakazakii ATCC 29004 | + |
| Cronobacter sakazakii BAA-894 | + |
| Cronobacter sakazakii ES15 | + |
| Cronobacter sakazakii ATCC 51329 | ++ |
| Cronobacter sakazakii isolates 1 | ++ |
| Cronobacter sakazakii isolates 2 | ++ |
| Cronobacter sakazakii isolates 3 | − |
| Cronobacter sakazakii isolates 4 | − |
| Cronobacter sakazakii isolates 5 | ++ |
| Cronobacter sakazakii isolates 6 | ++ |
| Cronobacter sakazakii isolates 7 | − |
| Cronobacter sakazakii isolates 8 | − |
| Cronobacter sakazakii isolates 9 | − |
| Cronobacter sakazakii isolates 10 | − |

++; 0.1 ≤ EOP ≤ 1;
+; EOP ≤ 0.1;
−; not susceptible to SG01

Referring to the results shown in Table 1, phage SG01 specifically infected Cronobacter sakazakii and did not cause infection in other strains.

Specifically, the phage exhibited activity against a broad range of Cronobacter sakazakii strains, including ten strains: ATCC 29544, NCTC 2949, ATCC 29004, ATCC BAA-894, ES15, ATCC 51329, isolates 1, isolates 2, isolates 5 and isolates 6. It was found to be capable of lysing approximately 63% of all tested Cronobacter sakazakii strains.

Accordingly, phage SG01 can selectively lyse Cronobacter sakazakii and is expected to be usefully applied in the food industry where control of Cronobacter sakazakii is required.

### Experimental Example 5: Measurement of Thermal and pH Stability of Phage SG01

The stability of phage SG01 was evaluated by measuring its survival rate across a temperature range of −18 to 80° C. and a pH range of 3 to 12.

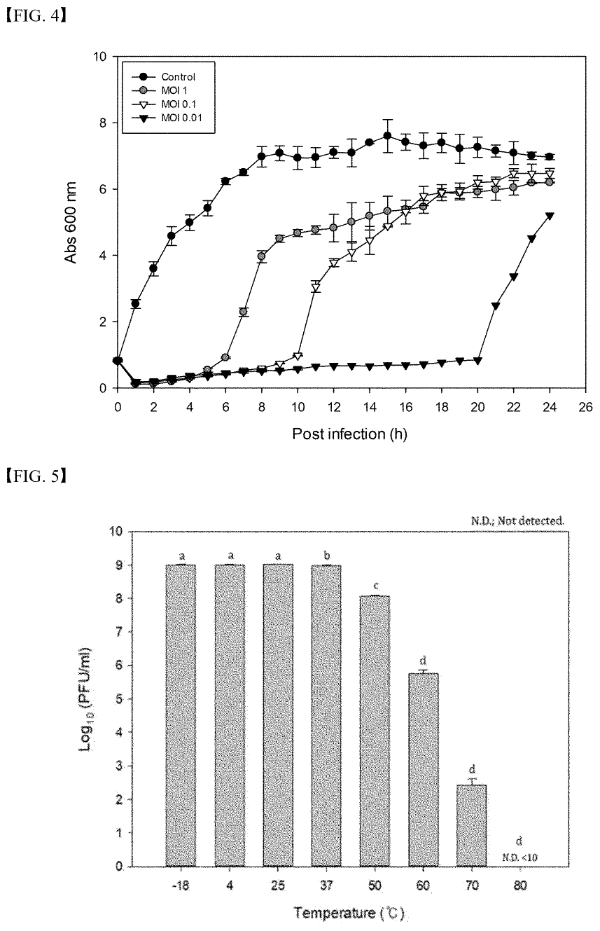

For thermal stability testing, a mixed solution of 990 μL of PBS buffer and 10 μL of phage SG01 was incubated for 30 minutes at temperatures of −18, 4, 25, 37, 50, 60, 70 and 80° C., and the remaining phage was quantified by plating. The experimental results are shown in FIG. 5.

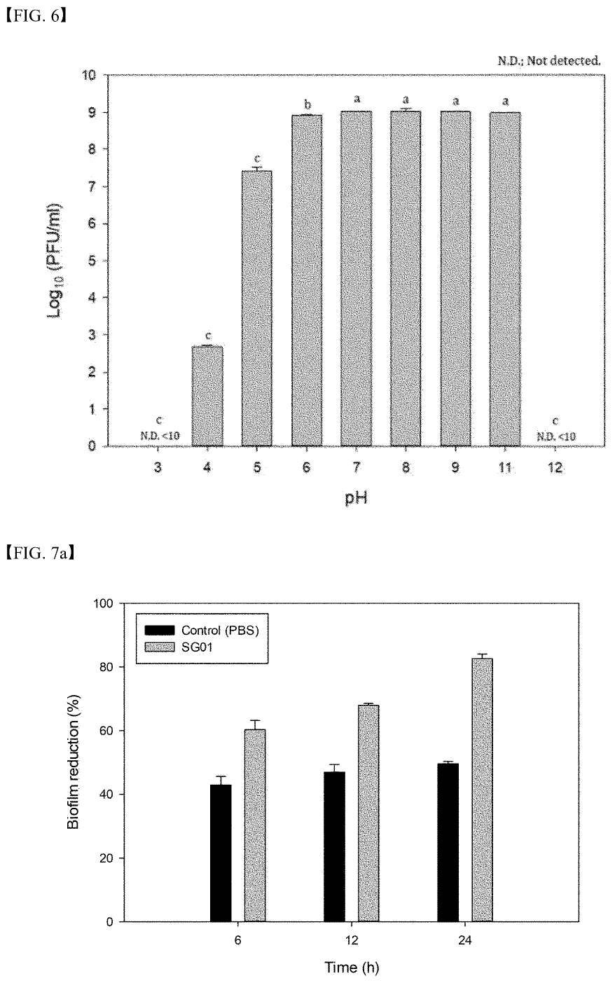

For pH stability testing, a mixed solution of 990 μL of pH buffer and 10 μL of phage SG01 was incubated in buffers with pH 3, 4, 5, 6, 7, 8, 9, 11 and 12 for 30 minutes, and the remaining phage was quantified by plating. The experimental results are shown in FIG. 6.

Referring to the thermal stability test results in FIG. 5, it was confirmed that the phage remained viable without complete loss of viability in the temperature range of −18 to 70° C. In particular, after 30 minutes of incubation at −18, 4, 25 and 37° C., the survival rate of the phage was barely affected, and most phage remained highly viable up to 50° C.

According to the pH stability test results shown in FIG. 6, the phage survived in the pH range of 4 to 11, and especially remained highly stable even after 30 minutes of incubation in the pH range of 6 to 11.

From these results, it was confirmed that phage SG01 can be usefully applied in foods and food manufacturing industries where thermal and pH stability are required.

### Experimental Example 6: Analysis of Biofilm Control Activity of Phage SG01

The biofilm control activity of phage SG01 was evaluated by measuring the biofilm reduction and biofilm inhibition of Cronobacter sakazakii in the phage SG01-treated group.

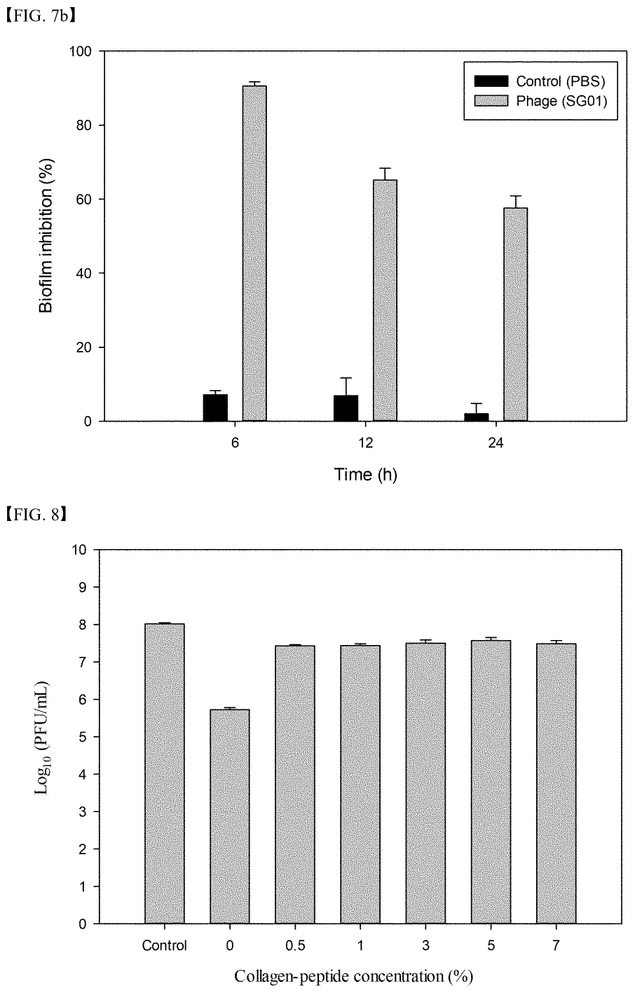

For biofilm reduction measurement, Cronobacter sakazakii ATCC 51329 was cultured in a 96-well plate for 48 hours to allow biofilm formation. Phage SG01 was then added to the culture at an MOI of 0.01, and after 6, 12 and 24 hours, the optical density at 600 nm was measured using a spectrophotometer. The biofilm reduction rate at each time point was calculated based on the measurement results, and the results are shown in FIG. 7a.

In addition, for measurement of biofilm inhibition rate, a mixture of Cronobacter sakazakii ATCC 51329 and phage was prepared by adding the phage at an MOI of 0.01, and the mixture was incubated in a 96-well plate for 6, 12, and 24 hours, respectively. The absorbance at 600 nm for each time point was measured using a spectrophotometer. The biofilm inhibition rate at each time point was calculated using the measured results and is shown in FIG. 7b.

Referring to FIG. 7a, in the sample treated with the phage after biofilm formation, the biofilm reduction rates at 6, 12 and 24 hours were approximately 60, 65 and 80%, respectively, whereas in the control group, the biofilm reduction rate remained low at around 40%.

In addition, referring to FIG. 7b, when the amount of biofilm formation in the phage-treated sample was assessed and the biofilm inhibition rate was calculated, the inhibition rates at 6, 12 and 24 hours were approximately 90, 65 and 60%, respectively, while in the control group, the biofilm inhibition rate was found to be less than 10%.

From these results, it was confirmed that treatment with phage SG01 can effectively control biofilm induced by Cronobacter sakazakii.

### Experimental Example 7: Phage Survivability Test During Freeze-Drying

Collagen peptide powder was sufficiently dissolved in an aqueous solution and sterilized by autoclaving to prevent contamination by other microorganisms.

To 10 mL of encapsulating solution containing collagen peptide, 100 μL (10⁹ PFU) of phage SG01 solution was added, and each mixed solution was frozen at −80° C. for at least 12 hours and subsequently freeze-dried at −50° C. for 72 hours to prepare encapsulated bacteriophage powder.

For the evaluation of survivability efficiency according to collagen peptide concentration, bacteriophage powder was prepared by the above method using encapsulating solutions containing collagen peptide at concentrations of 0.005, 0.01, 0.03, 0.05 and 0.07 g/mL.

Each bacteriophage powder was dissolved in 10 ml of PBS, and the number of bacteriophages (PFU/mL) released from the powder was measured. Based on the measurement results, the survivability of the bacteriophage was evaluated according to each collagen peptide concentration, and the results are shown in FIG. 8.

Referring to FIG. 8, when the bacteriophage was encapsulated using collagen peptide, the bacteriophage decreased by less than approximately 1 log even after freeze-drying, indicating that most of the phages remained viable. In contrast, in the absence of collagen peptide, the bacteriophage decreased by approximately 2 log or more, exhibiting low survivability. Among the tested conditions, the number of surviving phages was highest when the collagen peptide concentration was 0.05 g/mL.

From these results, it was confirmed that collagen peptide is suitable as a base material for encapsulating phages, and that using collagen peptide at a concentration of 0.05 g/mL is the most appropriate for phage encapsulation.

### Experimental Example 8: Trehalose Concentration Determination Test

Using the same method as in Experimental Example 7, collagen peptide (0.05 g/mL) was used as the encapsulating material, and trehalose was added at various concentrations as a cryoprotectant. The phage survivability was then compared.

To evaluate the survivability according to trehalose concentration, encapsulated bacteriophage powders were prepared using the following groups: experimental group A using only aqueous solution during freeze-drying; trehalose-only groups B to E, in which trehalose was used as the encapsulating solution at concentrations of 0.005, 0.01, 0.015 and 0.02 g/mL, respectively; collagen peptide-only group F, in which collagen peptide was used at 0.05 g/ml; and combined collagen peptide/trehalose groups G to J, in which collagen peptide at 0.05 g/mL was combined with trehalose at concentrations of 0.005, 0.01, 0.015 and 0.02 g/mL, respectively.

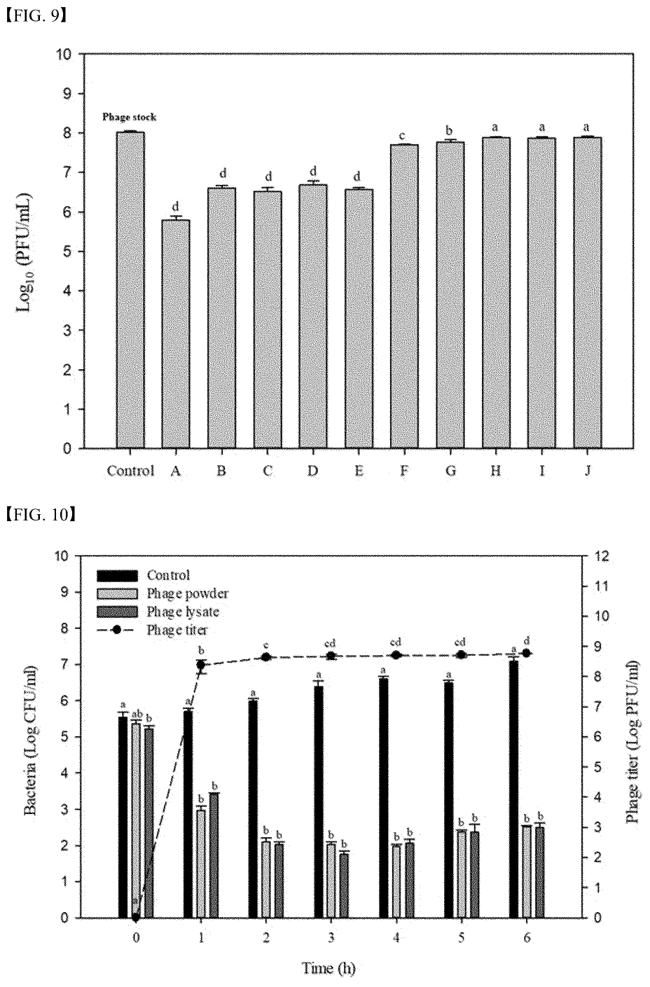

FIG. 9 shows the phage survivability after freeze-drying according to trehalose concentration. In the trehalose-only groups (B to E), the number of surviving bacteriophages decreased by approximately 1.5 log or more, whereas in the groups using both collagen peptide and trehalose, the decrease was less than approximately 1 log, indicating higher survivability. Among these, the highest phage survival was observed when 0.01 g/mL or more of trehalose was added to 0.05 g/mL collagen peptide.

These results confirmed that the combination of collagen peptide and trehalose exhibits a synergistic effect in improving phage survivability, and that the use of 0.01 g/mL trehalose in combination with 0.05 g/mL collagen peptide is the most appropriate for phage encapsulation.

### Experimental Example 9: Comparison of Antimicrobial Activity of Phage Solution and Phage Powder Against Bacteria in Infant Formula

To evaluate the antimicrobial activity of phage SG01 in food, phage SG01 solution and powder were added to powdered infant formula inoculated with Cronobacter sakazakii, and the bacterial count was measured.

To observe changes in the number of Cronobacter sakazakii cells in infant formula following phage treatment, 0.1 mL of Cronobacter sakazakii cell suspension was first inoculated into 2.9 mL of sterilized infant formula to adjust the final bacterial concentration to 10⁶ CFU/mL. The resulting mixture was then treated in two ways: with a phage solution (phage lysate) and with phage powder.

Using the method described in Experimental Example 7, an encapsulated bacteriophage powder was prepared with 0.05 g/mL of collagen peptide and 0.01 g/ml of trehalose. In the phage solution-treated group, 1 mL of phage solution (10⁸ PFU) prior to freeze-drying was added to the infant formula inoculated with bacteria. In the phage powder-treated group, 0.1 g of collagen peptide and trehalose-based phage powder (10⁸ PFU) was dissolved in 1 mL of PBS buffer and added.

All phage-treated groups were set at an MOI of 1,000, and the control group was prepared by adding 1 mL of PBS buffer only to the mixture. All groups were incubated at 25° C. for up to 6 hours. Every hour, 100 μL of each solution was collected, serially diluted, plated on TSA agar, and the remaining bacterial count (CFU/mL) was measured. In addition, at each time point, the sample was also serially diluted, mixed with 5 mL of 0.4% TSA soft agar and 100 μL of the diluted solution, and overlaid on TSA agar to measure the number of lytic phages (CFU/mL).

FIG. 10 shows the antimicrobial activity of liquid-phase and powder-phase phage against bacteria in infant formula. Referring to FIG. 10, in the control group without phage treatment, the bacterial count in infant formula gradually increased over time. In contrast, when liquid-phase or powder-phase phage was added, the bacterial count decreased by approximately 10² and 10³ CFU, respectively, compared to the initial level after 1 hour, and both phage types maintained effective antimicrobial activity for up to 6 hours. Furthermore, the phage release test confirmed that the phages were maximally released from the capsules within 1 hour, resulting in rapid antimicrobial activity.

In summary, while bacterial growth and reproduction continued in the absence of phage treatment, both liquid and powder forms of phage SG01 exhibited antimicrobial activity. The powder form maintained antimicrobial efficacy equivalent to that of the liquid form, even after phage powdering, and demonstrated rapid antimicrobial action due to the quick release of phages.

While certain embodiments of the present disclosure have been described above, the present disclosure is not limited to the embodiments as described. Rather, various modifications and variations may be made without departing from the spirit or scope of the present disclosure, and such modifications and variations are to be understood as falling within the scope of the technical idea of the present disclosure.

1. A bacteriophage having antibacterial activity against Cronobacter sakazakii, deposited under accession number KCTC15074BP.

2. The bacteriophage according to claim 1, wherein the bacteriophage belongs to family Siphoviridae.

3. The bacteriophage according to claim 1, wherein the bacteriophage is used at a multiplicity of infection (MOI) of 0.1 or less.

4. A method for preparing a bacteriophage powder, comprising:
(i) mixing an encapsulating solution comprising a collagen peptide and a bacteriophage to prepare a mixed solution; and
(ii) drying the mixed solution to obtain an encapsulated bacteriophage powder.

5. The method for preparing a bacteriophage powder according to claim 4, wherein the bacteriophage is a bacteriophage having antibacterial activity against Cronobacter sakazakii.

6. The method for preparing a bacteriophage powder according to claim 4, wherein the bacteriophage is the bacteriophage deposited under accession number KCTC 15074BP.

7. The method for preparing a bacteriophage powder according to claim 4, wherein a concentration of the bacteriophage in the mixed solution is 10⁵ to 10¹⁵ PFU/mL.

8. The method for preparing a bacteriophage powder according to claim 4, wherein the concentration of the collagen peptide in the encapsulating solution is 0.01 to 0.2 g/mL.

9. The method for preparing a bacteriophage powder according to claim 4, wherein the encapsulating solution further comprises trehalose.

10. The method for preparing a bacteriophage powder according to claim 9, wherein the concentration of the trehalose in the encapsulating solution is 0.001 to 0.1 g/mL.

11. The method for preparing a bacteriophage powder according to claim 4, wherein the mixed solution comprises one or more solvents selected from the group consisting of water, methanol, ethanol, propanol, 1,3-propanediol, butanol, pentanol, hexanol, propylene glycol, dipropylene glycol, butylene glycol, glycerin, acetone, ethyl acetate, butyl acetate, chloroform, diethyl ether, dichloromethane and hexane.

12. The method for preparing a bacteriophage powder according to claim 4, wherein step (ii) is performed by freezing the mixed solution at a temperature below 0° C. for 3 hours or longer and then drying it for 18 hours or longer.

13. A bacteriophage powder prepared by the method according to claim 4.

14. The bacteriophage powder according to claim 13, wherein the bacteriophage powder has a structure comprising a bacteriophage and a capsule wall, wherein the capsule surrounds the bacteriophage and comprises a collagen peptide.
